(19) United States
(12) Plant Patent Application Publication
(10) Pub. No.: US 2007/0289038 P1
(43) Pub. Date: Dec. 13, 2007

(54) Guinea impatiens plant named 'Ingcarmi'

(75) Inventor: Monica Maria Adelheid Sanders, Grootebroek (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/448,233

(22) Filed: Jun. 7, 2006

(51) Int. Cl. A01H 5/00

(52) U.S. Cl. PLT/318

(57) ABSTRACT

A new and distinct cultivar of New Guinea Impatiens plant named ‘Ingcarmi’ characterized by its large magenta colored flowers with dark green foliage, early flowering, good heat tolerance, nice rounded shaped growth habit with excellent basal branching and good vigor.

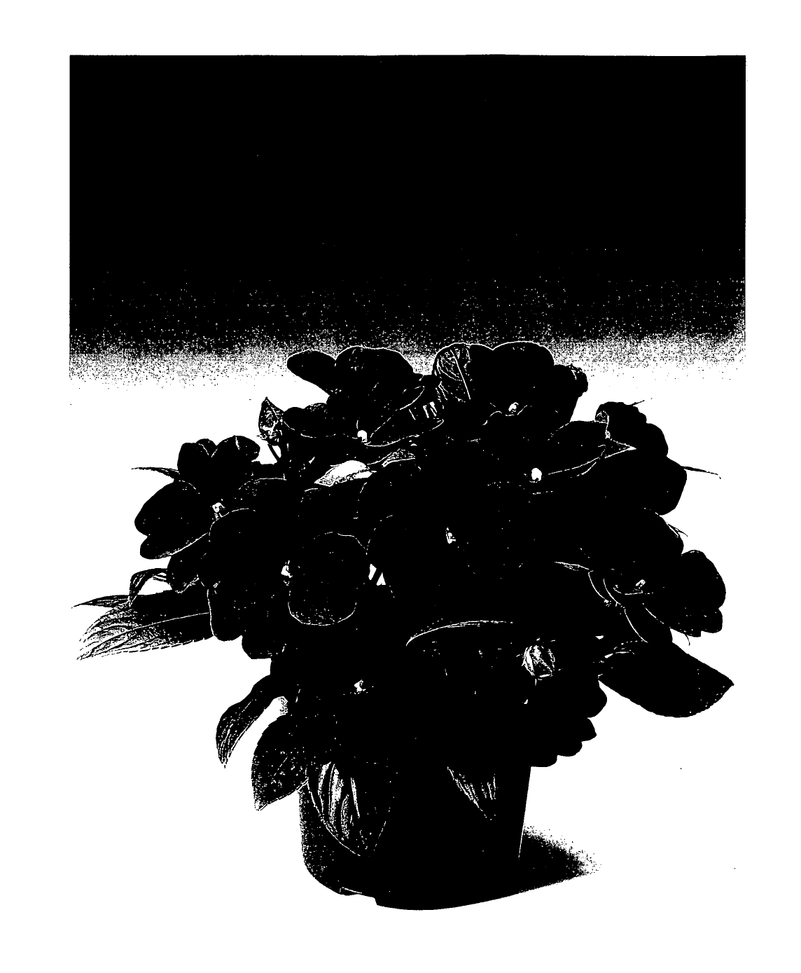

### LATIN NAME OF THE GENUS AND SPECIES OF THE PLANT CLAIMED

Impatiens hawkeri

### VARIETAL DENOMINATION

‘Ingcarmi’

### BACKGROUND OF THE NEW PLANT

The present invention comprises a new and distinct New Guinea Impatiens plant botanically known as Impatiens hawkeri and referred to by the cultivar name ‘Ingcarmi.’

The new cultivar was developed in a controlled breeding program conducted by the inventor in Enkhuizen, Netherlands. The New Guinea Impatiens cultivar is very floriferous with large flowers, early flowering on a strong growing, basal branching plant with good heat tolerance.

The new cultivar is propagated from cuttings resulting from the cross in Sep. 2000 of the proprietary New Guinea Impatiens selection identified as ‘G576-1’ as female parent and the proprietary New Guinea Impatiens selection identified as ‘G17-1’ as male parent. ‘G576-1’ is not commercially available and has not been patented. ‘G17-1’ is not commercially available and has not been patented.

As a result of this cross the present cultivar was selected in Sep. 2001 in Enkhuizen, Netherlands and has been repeatedly asexually reproduced by cuttings in Enkhuizen, Netherlands, in Gilroy, Calif., and in Angers, France over a period of several years. The distinctive characteristics of this new Impatiens are stable and reproduced true to type in successive generations of asexual reproduction. It takes 8 to 10 weeks to produce a finished plant, starting from a rooted plug and planted in a 12 cm pot, depending on the temperature.

### SUMMARY OF THE INVENTION

The following traits have been repeatedly observed and are determined to be the unique characteristics of ‘Ingcarmi.’ These characteristics in combination distinguish ‘Ingcarrni’ as a new and distinct New Guinea Impatiens cultivar:
1. Large magenta flowers
2. Early flowering and floriferous habit
3. Rounded shaped habit
4. Strong growing and good heat tolerance

Plants of the New Guinea Impatiens named ‘Ingcarmi’ differ primarily from the plants of the female parent selection in the following characteristic:

Plants of the New Guinea Impatiens named ‘Ingcarmni’ have dark green foliage, whereas the plants of the female parent selection have lighter green foliage.

Plants of the New Guinea Impatiens named ‘Ingcarmi’ differ primarily from the plants of the male parent selection in the following characteristic:

Plants of the New Guinea Impatiens named ‘Ingcarmi’ have a magenta flower color, whereas the plants of the male parent selection have a rose flower color.

Plants of the New Guinea Impatiens named ‘Ingcarmi’ can be compared to plants of ‘Fisimp 551,’ U.S. Plant Pat. No. 13,702. In side-by-side comparisons conducted in Enkhuizen, Netherlands, plants of the New Guinea Impatiens named ‘Ingcarmi’ differed from plants of the cultivar ‘Fisimp 551’ in the following characteristics:
1. Plants of the New Guinea Impatiens named ‘Ingcarmi’ have large magenta flowers, whereas plants of ‘Fisimp 551’ have carmine red flowers.
2. Plants of the New Guinea Impatiens named ‘Ingcarmi’ have medium sized, dark green foliage, whereas plants of ‘Fisimp 551’ have large, bronze foliage.
3. Plants of the New Guinea Impatiens named ‘Ingcarmi’ have a good branching habit, whereas plants of ‘Fisimp 551’ have an upright growing habit.

### DESCRIPTION OF THE DRAWING

The New Guinea Impatiens named ‘Ingcarmi’ plant is illustrated by the accompanying photographic drawing which shows blooms, buds and foliage of the plant in full color, the color showing being as true as can be reasonably obtained by conventional photographic procedures.

### DESCRIPTION OF THE NEW CULTIVAR

The following detailed descriptions set forth the distinctive characteristics of the New Guinea Impatiens named ‘Ingcarmi’. The data which defines these characteristics were collected from asexual reproductions carried out in Enkhuizen, Netherlands. The plant history was taken on 30 week old plants, blossomed under natural light in the field. Color readings were taken in the laboratory under ambient light. Color references are primarily to the RHS Color Chart of The Royal Horticultural Society of London.
The plant:
   Classification.—Botanical: Impatiens hawkeri.
Parentage:
   Female parent.—Proprietary Impatiens hawkeri selection identified as code number ‘G576-1,’ not patented.
   Male parent.—Proprietary Impatiens hawkeri selection identified as code number ‘G17-1,’ not patented.
Propagation:
   Type cutting.—Terminal cuttings.
   Time to initiate roots.—Approximately 7-14 days.
   Time to produce a rooted cutting.—Approximately 21 days.
   Root description.—Fine, fibrous, and white in color.
Plant description:
   General appearance.—Outwardly spreading, low rounded and uniform mounded plant growth.
Habit:
   Growth and branching habit.—Freely branching habit, freely flowering, moderate vigorous to vigorous.
Crop time:
   Plant height.—25-30 cm.
   Plant spread.—44-50 cm.
Lateral branch description:
   Length.—25-27 cm.
   Diameter.—4-6 mm.
   Texture.—Smooth, glabrous.
   Internode length.—2-6 cm.
   Color.—RHS 187A.
   Quantity of lateral branches.—16-18.
Foliage description:
   Arrangement.—Primarily in whorls.
   Length, mature leaves.—8.5-9.5 cm.
   Width, mature leaves.—2.3-3 cm.
   Shape.—Lanceolate.
   Apex.—Acute to acuminate.
   Base.—Acute.
   Margin.—Serrulate with fine cilation.
   Texture of upper surface.—Smooth, glabrous.
   Texture of lower surface.—Smooth, glabrous.
   Venation pattern.—Pinnate.
   Color young foliage, upper surface.—RHS 137A.
   Color young foliage, lower surface.—RHS 147C.
   Fully expanded foliage, upper surface.—RHS 139A.
   Fully expanded foliage, lower surface.—RHS 148B.
   Venation, upper surface.—RHS 151C.
   Venation, lower surface.—RHS 183B.
   Anthocyanin.—Present in veins of lower surface, not present in veins of upper surface.
   Petiole length.—About 1.5-2.5 cm.
   Petiole diameter.—About 3.5 mm.
   Texture of petiole.—Smooth, glabrous.
   Color of petiole.—RIS 185A.
Flower description:
   Flower type and flowering habit.—Single.
   Number of flowers per leaf axil.—1-3.
   Number of flowers and flower buds per lateral branch.—7.
   Flower position.—Above and beyond the foliage and typically facing upwards and outward.
   Flower shape.—Rounded, mostly flat.
   Flowers lasting.—About 10 days on the plant depending on environmental conditions.
   Petals.—Self cleaning.
   Gynoecium.—Persistent.
   Flowers.—Not fragrant.
   Flowering.—Indeterminate and continuous.
   Flowering season.—Year round under greenhouse conditions. In the garden, flowering from spring until fall.
   Flower length.—About 5.5-6.5 cm.
   Flower width.—About 5-6 cm.
   Flower depth.—About 0.2 cm.
Flower buds:
   Bud length.—About 1.5 cm.
   Bud diameter.—About 1 cm.
   Bud shape.—Ovoid.
   Texture.—Smooth, glabrous.
   Color, just before opening.—RHS 60A.
Petals:
   Quantity.—Single, five per flower, imbricate.
   Length of banner petal.—About 2.7 cm
   Width of banner petal.—About 4.2 cm.
   Length lateral and basal petals.—About 3 cm.
   Width lateral and basal petals.—About 2.7 cm.
   Lateral and basal petal shape.—Obcordate.
   Banner petal shape.—Obcordate.
   Petal apex.—Retuse to emarginate.
   Petal base.—Attenuate.
   Petal margin.—Entire.
   Petal texture of upper surface.—Smooth, satiny.
   Petal texture of lower surface.—Smooth.
   Petal color.—Upper surface at top of all petals: When opening and fully opened: RHS 45B. — Upper surface at base of all petals: When opening and fully opened: RHS N66A — Lower surface: When opening and fully opened: RHS 53D.
Spur:
   Number of spurs per flower.—1
   Length.—About 3.5-4 cm.
   Spur diameter at apex.—1 mm.
   Spur diameter at flower.—About 3 mm.
   Texture.—Smooth, glabrous.
   Aspect.—Curved.
   Color.—RHS 60A.
Peduncles:
   Length.—About 5-7 cm.
   Strength.—Strong, flexible.
   Diameter.—0.2 cm.
   Aspect.—About 450 from the stem.
   Color.—RHS 185A.
Reproductive organs:
   Androecium.—Stamen number: Five fused at anthers, filaments free, hooded.
   Anther shape.—Obovate.
   Anther length.—About 3.5 mm.
   Anther color.—MHS 65D, near filament RHS 45C.
   Amount of pollen.—Scarce to moderate.
   Color of pollen.—RHS 158A.
Gynoecium:
   Pistil number.—One.
   Pistil length.—About 5 mm.
   Stigma shape.—Columnar, five segmented.
   Stigma color.—Colorless.
   Style color.—RHS 154D.
   Ovary arrangement.—Five celled.
   Ovary color.—RHS N144C.
Seed development: Seed development has not been observed.
Disease and pest resistance: Resistance to pathogens and pests common to Impatiens has not been observed.

What is claimed is:

1. A new and distinct cultivar of New Guinea Impatiens plant named ‘Ingcarmi’, as substantially illustrated and described herein.
